(19) United States
(12) Patent Application Publication
(10) Pub. No.: US 2006/0210613 A1
(43) Pub. Date: Sep. 21, 2006

(54) Therapeutic wound care product

(76) Inventor: Richard Daniel Carliss, Mobile, AL (US)

Correspondence Address: DOCKET ADMINISTRATOR;LOWENSTEIN SANDLER PC, 65 LIVINGSTON AVENUE, ROSELAND, NJ, 07068, US

(21) Appl. No.: 11/375,775

(22) Filed: Mar. 14, 2006

Related U.S. Application Data
(60) Provisional application No. 60/661,423, filed on Mar. 15, 2005.

(51) Int. Cl. A61K 31/485; A61F 13/02

(52) U.S. Cl. 424/448; 514/282

(57) ABSTRACT

The present invention relates to a multi-layered product having at least one therapeutic agent therein and a layer containing a chitin or chitin derivative for treatment of a wound, and also to a method using such product.

### CROSS-REFERENCE TO RELATED APPLICATION

This application claims benefit of U.S. Provisional Application No. 60/661,423 filed Mar. 15, 2005, which is hereby incorporated by reference in its entirety.

### FIELD OF THE INVENTION

The present invention relates to a multi-layered product having at least one therapeutic agent therein and a layer containing a chitin or chitin derivative for treatment of a wound and also to a method using such product.

### BACKGROUND OF THE INVENTION

Skin wounds can be treated locally by the topical administration of a therapeutic agent directly to the skin. For example, pain resulting from a skin wound can be treated with use of an anesthetic and/or analgesic resulting in the blockade of nociceptive and other stimuli. Locally applied analgesics and/or anesthetics have been shown to prevent the generation and conduction of nociceptive nerve impulses. Local anesthetics or sodium-channel blockers, such as lidocaine, prevent the generation and conduction of nerve impulses by decreasing or preventing the large transient increase in the permeability of excitable membranes to sodium ion (Na⁺).

A wound that penetrates the integument, for example, a surface wound, provides an entry mechanism for the deeper diffusion of a therapeutic agent. Examples of surface wounds include those resulting from an abrasion, bum, tear or other penetrating insult. One means of delivery of the therapeutic agent is by use of a bandage-type product. The use of self-adhering bandage products that have an adhesive, such as a pressure sensitive adhesive, that allows the product to be held in place without additional means, have enjoyed popular success. For example, BAND-AID® Brand Adhesive Bandages (Johnson & Johnson, New Brunswick, N.J., U.S.A.) have been commercially available for a number of years, in a number of sizes, with a popular size being ¾ inch wide by 3 inches long, and as individually wrapped units packaged in boxes of designated quantities.

The use of self-adhesive bandage-type products for the delivery of therapeutic agents is also known in the medical arts. For example, U.S. Pat. No. 3,797,494 teaches a bandage having a backing member, an adhesive surface and between them a reservoir containing a drug, for administration to the skin or mucosa; an adhesive bandage strip having a rupturable pocket confining a medicament and self-contained means for rupturing the pocket is taught in U.S. Pat. No. 4,117,841; and U.S. Pat. No. 4,858,604 teaches an adhesive bandage having a medicine sealed in a blister with a medicine covering film interposed between the blister and an absorbent pad on an adhesive tape, where the medicine is released into the pad when pressure is applied to the blister. Patches as the adhesive bandage-type product are also well known in the art.

Other bandage products in the over-the-counter market contain small amounts of capsaicin, which is painful to open integumental wounds and largely ineffective in providing pain relief. See also, US Patent Application Publication No. 2001/0002406. Bandages have also been disclosed that produce either heat or cold for wound care treatment. See, for example., U.S. Pat. No. 5,658,583 and U.S. Patent Application Publication Nos. 2005/0080368 and 2005/0085751. Yet such bandages do not directly address wounds of the integument such as abrasions. Also, no haemostatic components are found in these products other than applied pressure. U.S. Pat. Nos. 6,897,348 and 6,967,261 teach a bandage for acute wounds and burns having an antimicrobial agent and a haemostatic agent such as chitosan.

Thus, there is a need for a dual-purpose product that can deliver a therapeutic agent to an integumental wound, and has also a haemostatic agent to staunch or keep in check the flow of blood from the wound. Such a product would allow for the delivery of the therapeutic agent to the superficial level of the peripheral nervous system, thereby enhancing the effect of the therapeutic agent.

### SUMMARY OF THE INVENTION

The present invention provides a product having a base layer, a first internal layer, and a second internal layer. The base layer has an exterior surface and an interior surface. The first internal layer of the product has a top face, a bottom face, an adhesive, and a reservoir. The bottom face of the first internal layer adheres to the interior surface of the base layer by virtue of the adhesive. The reservoir is housed between the top and bottom faces of the first internal layer and has at least one therapeutic agent; and the second internal layer has a chitin or a chitin derivative, a top face and a bottom face. The bottom face of the second internal layer adheres to the top face of the first internal layer. The present invention further provides a product containing at least one therapeutic agent selected from the group consisting of anesthetics, analgesics, antifungals, antibiotics, antipruritics, and antipyretics. The therapeutic agent is housed in the reservoir portion of the product.

The present invention also provides a method of treating a wound by affixing to the area surrounding the wound the product of the invention thereby, covering the wound.

### DETAILED DESCRIPTION OF THE INVENTION

In one aspect, the present invention relates to a product having multiple layers, including a base layer, first internal layer and second internal layer, where the first internal layer has at least one therapeutic agent and the second internal layer includes chitin or a derivative thereof.

In some embodiments, the product of the present invention has:

(1) a base layer;

(2) a first internal layer; and

(3) a second internal layer,

wherein:

the base layer has an exterior surface and an interior surface;

the first internal layer has:

(1) a top face;

(2) a bottom face;

(3) an adhesive; and

(4) a reservoir;

wherein:

the bottom face of the first internal layer adheres to the interior surface of the base layer by virtue of the adhesive; and

the reservoir is housed between the top and bottom faces of the first internal layer and comprises at least one therapeutic agent; and

the second internal layer has:

(1) a chitin or a chitin derivative;

(2) a top face; and

(3) a bottom face,

wherein the bottom face of the second internal layer adheres to the top face of the first internal layer.

As used herein, “product” refers to a means of topically covering a wound or condition. The product thereby shields the wound or condition from the external environment, contributing to the healing or improvement of the wound or condition. The product can be of various forms, for example, and without limitation, a bandage, dressing, patch, pad, tape or wrap.

“Reservoir,” as used herein, refers to the place and system where the therapeutic agent is located. In some embodiments of the product of the invention, the reservoir contains at least one therapeutic agent selected from the group consisting of anesthetics, analgesics, narcotics, anti-inflammatory agents, cyclooxygenase inhibitors, antibiotics, antifungals, antineoplastics, astringents, antipruritics, antipyretics, and antihistamines. In some such embodiments, the reservoir further includes a support matrix that contains the therapeutic agent.

A schematic illustration of an embodiment of the product of the invention is shown below. In such an embodiment, the product of the invention has a base layer (A), a first internal layer (B), and a second internal layer (C). The first internal layer (B) has a top face, a bottom face, an adhesive and a reservoir, and adheres to the interior surface of the base layer by virtue of the adhesive while the reservoir is housed between its top and bottom faces and contains at least one therapeutic agent. The second internal layer (C) contains a chitin or a chitin derivative.

[image]

The support matrix of the reservoir provides a physical means in which the therapeutic agent is stored. The support matrix can be a solid, liquid, or gas or some state there between, for example, a semi-solid. The support matrix can be a mixture of two or more substances such as a solution, powder or colloid such as a suspension (dispersion), emulsion, sol, gel, foam. Various substances that are useful as the support matrix include, but are not limited to, aqueous solvents, non-aqueous solvents, carbohydrates such as polysaccharides, elastomers such as silicone elastomers, fatty acids, fibrous materials such as cotton, wool, and woven and non-woven textiles, polymers such as silicones, polyesters, vinyl polymers, acrylic polymers such as methacrylates, cellulose ethers, polysaccharides and related polymers, triglycerides, waxes, and oils. Representative, non-limiting examples include, 7-dehydrocholesterol, abietic acid, acetylated glycol stearate, agar, aluminum/magnesium hydroxide stearate, aluminum caprylate, aluminum dilinoleate, aluminum dimyristate, aluminum distearate, aluminum isostearate, aluminum isostearates/laurates/palmitates, aluminum isostearates/laurates/stearates, aluminum isostearates/myristates, aluminum isostearates/palmitates, aluminum isostearates/stearates, aluminum myristates/palmitates, aluminum stearates, aluminum tristearate, avocadamide DEA, bentonite, beta-sitosterol, C₁₂₋₁₃ alcohols, C₁₂₋₁₅ alcohols, C₁₂₋₁₆ alcohols, C₁₅₋₁₈ glycols, C₂₂₋₂₄ pareth-33 C₉₋₁₁ alcohols, calcium carrageenan, callitris quadrivalvis, carbomer, carboxymethyl hydroxyethylcellulose, carboxymethyl hydroxypropylcellulose, guar, carrageenan, cellulose gum, cera microcristallina, ceresin, cetearyl alcohol, chitin, chitin derivatives such as chitosan, cocamide, cocamide DEA, cocamide MEA, cocamide MIPA, cocamidopropyl lauryl ether, collagen, a collagen/pectin matrix, collodion, cotton, cyanopsis tetragonalba, dihydroxyethyl cocamine oxide, ethylene/acrylic acid copolymer, ethylene/VA copolymer, gelatin, glycol cetearate, gum Arabic, hydrogenated C₁₂₋₁₈ triglycerides, hydrogenated lanolin alcohol, hydrogenated tallow amide, hydroxybutyl methylcellulose, hydroxyethyl ethylcellulose, hydroxyethylcellulose, hydroxypropyl guar, hydroxypropyl methylcellulose, hydroxypropylcellulose, isopropyl ester of PVM/MA copolymer, lanolinamide DEA, latex, lauryl alcohol, maltodextrin, povidone, iodinic povidone, methoxy PEG-22/dodecyl glycol copolymer, methylcellulose, microcrystalline cellulose, montmorillonite, mucopolysaccharide, myristyl alcohol, nylon, oxyquinoline sulfate, ozokerite, palm kernelamide DEA, palm kernelamide MEA, palm kernelamide MIPA, palmamide DEA, palmamide MEA, palmamide MIPA, peanutamide MEA, peanutamide MIPA, pectin, Peg-115m, Peg-14m, Peg-20m, Peg-23m, Peg-2m, Peg-45/dodecyl glycol copolymer, Peg-5m, Peg-7m, Peg-90m, Peg-9m, pentadecyl alcohol, phenyl-formaldehyde resins, polyacrylic acid, polydimethylsiloxane, polyethylene, polyurethane, polyvinyl acetate, potassium alginate, potassium carrageenan, PVM/MA copolymer, PVP, PVP/dimethylaminoethylmethacrylate copolymer, PVP/VA copolymer, rayon, saccharated lime, scleroglucan, sclerotium gum, silicone elastomer, silicone oxide, sodium acrylate/vinyl alcohol copolymer, sodium c4-12 olefin/maleic acid copolymer, sodium carboxymethyl dextran, sodium carrageenan, sodium cellulose sulfate, sodium polymethacrylate, sodium polynaphthalenesulfonate, sodium polystyrene sulfonate, soyamide DEA, starch, stearic acid, stearyl alcohol, styrene/MA copolymer, sulfated polysaccharide, synthetic beeswax, synthetic candelilla wax, synthetic camauba, synthetic japan wax, synthetic wax, Tallow amide, Tallowamide DEA, Tallowamide MEA, tetrasodium etidronate, tridecyl alcohol, wool, and xanthan gum.

In some embodiments, the support matrix of the reservoir comprises a mixture selected from the group consisting of a solution, a powder and a colloid. In some embodiments, the solution is a collodion. Collodion is a clear or slightly opalescent, highly flammable, syrupy liquid compounded of pyroxylin, ether and alcohol, which dries to a transparent, tenacious film. Collodion has a wide range of uses in industry including, for example, applications in the manufacture of photographic film, in fibers, in lacquers, and in engraving and lithography. In medicine, collodion is used as a topical protectant, applied to the skin to close small wounds, abrasions, and cuts, to hold surgical dressings in place, and to keep medications in contact with the skin. Collodion also includes, flexible collodion, salicylic acid collodion and pyroxylin solutions. Flexible collodion is a preparation of collodion, camphor, and castor oil. Salicylic acid collodion is a preparation containing 9.5-11.5% salicylic acid in flexible collodion. Pyroxylin solutions consisting of nitrocellulose in ether or acetone with the addition of alcohols.

In some embodiments, the mixture of the support matrix is a powder. In some such embodiments, the powder is micronized.

In some embodiments, the colloid is a suspension, emulsion, sol, gel, or foam. In some embodiments, the colloid is a gel. In some such embodiments, the gel comprises at least one silicone polymer, gelatin, pectin, collagen, polyacrylamide, ethylene dimethacrylate polyethylene glycol dimethacrylate, glycol dimethacrylate or acemannan. Acemannan is a mannose polymer obtained from the mucilage of the Aloe vera plant. As used herein, “gel” refers to a colloid produced by combining a discontinuous phase (i.e., a dispersed phase) with a continuous phase (i.e., a dispersion medium or matrix) to produce a viscous, jelly-like, semisolid material. When water is the dispersion medium the gel is often referred to as a hydrogel.

In some embodiments, the support matrix is a fibrous substance. In some embodiments, the fibrous substance is selected from cellulose, cotton, modal, nylon, rayon or wool. As used herein, “fibrous substance” refers to any material having, resembling, and/or pertaining to fibers. Non-limiting, representative examples of fibrous substances include cellulose, cotton, modal, nylon, rayon, polyester fiber, acrylic fiber, aramide fiber, elastane (spandex) or wool.

In some embodiments, the support matrix has at least one gel, emulsion, collodion, or fibrous substance. As used herein, “emulsion” refers to a colloid system in which both the dispersed phase and the dispersion medium are immiscible liquids with the dispersed liquid being the discontinuous phase and the dispersion medium the continuous phase, whereby the dispersed liquid is distributed in small globules throughout the body of the dispersion medium liquid. Common types of emulsions are oil-in-water where oil is the dispersed liquid and an aqueous solution such as water is the dispersion medium and water-in-oil where conversely, an aqueous solution is the dispersed phase.

In some embodiments, the reservoir of the product of the invention further includes at least one stabilizer, emulsifier, thickening agent, solubilizing agent, antioxidant, colorant, surfactant, or control release agent.

“Stabilizer,” as used herein, is an agent that aids a composition to remain within its physical, chemical, microbiological, therapeutic, and toxicological specifications, and includes for example, and without limitation, emulsifiers, antioxidants and preservatives.

“Emulsifiers” or “emulsifying agent,” as used herein, promote the formation and stabilization of an emulsion. Suitable emulsifiers for the present invention may be natural materials, finely divided solids, or synthetic materials. Natural emulsifying agents may be derived from either animal or vegetable sources. Emulsifying agents from animal sources include, but are not limited to, gelatin, egg yolk, casein, wool fat, and cholesterol; those from vegetable sources include, but are not limited to, acacia, tragacanth, chondrus, pectin and cellulose derivatives. Cellulose derived emulsifiers include, but are not limited to, methyl cellulose and carboxymethyl cellulose, and are often used to increase viscosity. Finely divided emulsifiers include, but are not limited to, bentonite, magnesium hydroxide, aluminum hydroxide, and magnesium trisilicate. Synthetic emulsifying agents include, but are not limited to, anionic, cationic, or nonionic agents such as sodium lauryl sulfate, benzalkonium chloride, polyethylene glycol 400 monostearate and any combinations thereof.

“Thickeners” or “thickening agents,” as used herein, refer to agents that increase the density or viscosity of a mixture to which it is added. Suitable thickeners that can be used in the present invention include, but are not limited to, non-ionic water-soluble polymers such as hydroxyethylcellulose (commercially available under the trademark Natrosole® 250 or 350), cationic water soluble polymers such as Polyquat 37 (commercially available under the trademark Synthalen® (CN), fatty alcohols, fatty acids, anionic polymers and their alkali salts, and mixtures thereof.

As used herein, “solublizing agents” are those substances that enable a solute to dissolve in a medium in which the solute is otherwise insoluble. Representative examples of solublizing agents that are suitable in the present invention include, without limitation, complex-forming solublizers such as citric acid, EDTA, sodium meta-phosphatate, succinic acid, urea, cyclodextrin, polyvinylpyrrolidone, diethylammonium-ortho-benzoate, and micelle forming solubilizers such as TWEEN® polysorbates (e.g., TWEEN 80® and TWEEN 60® and Span sorbitan esters (e.g., sorbitan monostearate (Span 60) and sorbitan monoleate(Span 80)). Other solublizers that can be used in the present invention are, for example, polyoxyethylene sorbitan fatty acid ester, polyoxyethylene n-alkyl amine n-oxides, polyoxamers, organic solvents, such as acetone, phospholipids, and cyclodextrins.

An “antioxidant” is a substance capable of inhibiting or delaying oxidation or inhibiting or delaying reactions promoted by oxygen or peroxides. Representative, non-limiting examples of antioxidants that can be used in the present invention are ascorbic acid (Vitamin C), bio-flavonoids including those sourced from, e.g., grapes, cocoa, and green tea, butylated hydroxyanisole (BHA), butylated hydroxytoluene (BHT), hypophosphorous acid, coenzyme Q10, melatonin, potassium metasulfite, retinal (Vitamin A), resveratrol, selenium, sodium bisulfite, sodium metabisulfite, and Vitamin E, including tocotrienol and tocopherol, and any other derivatives of each of the foregoing.

A “preservative” is a substance that inhibits or prevents microbial growth. Representative, non-limiting examples of antioxidants that can be used in the present invention are alcohol, benzalkonium chloride, benzethonium chloride, benzyl alcohol, chlorobutanol, dehydroacetic acid, phenol, phenylethyl alcohol, phenylmercuric nitrate, potassium benzoate, potassium sorbate, sassafras oil, sodium benzoate, sorbic acid, and thimerosal.

“Colorant,” as used herein, is a substance that is used to give or change color, and includes pigments or dyes or a combination thereof. Suitable pigments for use in the present invention, include, without limitation, iron oxides and titanium oxides, while suitable dyes include FD&C approved colorants, D&C approved colorants, and those approved for use in Europe and Japan. See Marmion, D.N., Handbook of U.S. Colorants for Food, Drugs, Cosmetics, and Medical Devices, 3^{rd} ed., 1991 (John Wiley & Sons, New York), incorporated herein by reference.

“Surfactants,” also often called “surface-active agents,” are substances that exert a change on the surface properties of a liquid, especially one that reduces the liquid's surface tension, such as a detergent. Suitable surfactants for use with the present invention include, but are not limited to, sarcosinates, glutamates, sodium alkyl sulfates, ammonium alkyl sulfates, ammonium alkyleth sulfates, ammonium laureth-n-sulfates, sodium laureth-n-sulfates, isothionates, glycerylether sulfonates, sulfosuccinates and combinations thereof. In some embodiments of the present invention, the c6mposition includes an anionic surfactant selected from the group consisting of sodium lauroyl sarcosinate, monosodium lauroyl glutamate, sodium alkyl sulfates, ammonium alkyl sulfates, sodium alkyleth sulfates, and combinations thereof.

Other stabilizers, emulsifiers, preservatives, antioxidants, colorants, thickeners, solubilizing agents and surfactants suitable for the present invention are available to those in the art in; for example, Remington: The Science and Practice of Pharmacy, 20^{th} ed. (Gennaro, A. R., et al., eds.) Lippincott Williams & Wilkins: Philadelphia (2000), the contents of which are hereby incorporated by reference into the present application.

The reservoir of the product of the invention contains at least one therapeutic agent.

In some such embodiments, the therapeutic agent includes at least one anesthetic. An anesthetic is a drug or agent that is used to reduce or abolish the sensation of pain. Such an agent that whose anesthetic action is limited to an area of the body determined by the site of its application, whereby it produces its effect by blocking nerve conduction, is a local anesthetic. When the anesthetic action is a systemic effect (i.e., throughout the body), such an agent is commonly referred to as a general anesthetic. Suitable local anesthetics for the present invention include, without limitation, benzocaine, bupivacaine, cocaine, eucaine, lidocaine, levobupivacaine, oxybupricaine, phenacaine, prilocaine, procaine, ropivacaine and tetracaine.

In some embodiments of the invention, the anesthetic comprises at least one local anesthetic or a pharmaceutical salt thereof. In some such embodiments, the local anesthetic comprises at least one selected from lidocaine, benzocaine, procaine, cocaine, eucaine, prilocaine, tetracaine, oxybupricaine, and phenacaine, or a pharmaceutically acceptable salt of thereof. In some embodiments, the local anesthetic is lidocaine.

In some embodiments, the anesthetic of the product of the invention is in an amount from about 1% to about 10% (i.e., on a total therapeutic agent basis). In some embodiments, the amount of anesthetic is from about 1% to about 8%; from about 1% to about 5%; or from about 1% to about 3%. In some embodiments, the amount of anesthetic is in an amount of about 1%; about 2%; about 3%; about 4%; about 5%; about 6%; about 7%; about 8%; about 9%; or about 10%. In some embodiments, the amount of anesthetic is in an amount about less than 10%; less than 9%; less than 8%; less than 7%; less than 6%; less than 5%; less than 4%; less than 3%; less than 2%; or less than 1%.

In some embodiments, the therapeutic agent of the reservoir is at least one analgesic.

An “analgesic,” as used herein, is an agent that alleviates pain without causing loss of consciousness, including opioid or non-opioid analgesics. An “opioid analgesic,” as used herein, refers to a compound that binds with a number of closely related specific receptors (opioid receptors) in the central nervous system to block the perception of pain or affect the emotional response to pain. Representative, non-limiting examples of opioid analgesics include morphine, codeine, hydrocodone, hydromorphone, oxycodone, oxymorphone, nalbuphine, naloxone, naltrexone, buprenorphine, butorphanol, etorphine, methadone, levo-alphacetylmethadol (LAAM), pethidine (meperidine), fentanyl, alfentanil, sufentanil, remifentanil, ketobemidone, carfentanyl, propoxyphene , dextropropoxyphene, dextromoramide, bezitramide, piritramide, pentazocine, phenazocine, buprenorphine, butorphanol, nalbufine, dezocine, etorphine, tilidine, tramadol, loperamide, and diphenoxylate. Conversely, a “non-opioid analgesic,” as used herein, refers to a compound devoid of opioid receptor liabilities, though having a central nervous system and/or peripheral system effect. Representative, non-limiting examples of non-opioid analgesics include nonsteroidal anti-inflammatory (NSAIDs) drugs, including the arylalkanoic acids such as indomethacin, sulindac and diclofenac, the 2-arylpropionic acids (Profens) such as ibuprofen, ketoprofen, naproxen, ketorolac, carprofen and fenoprofen, the cyclooxygenase (COX) inhibitors (Coxibs; e.g., diaryl-substituted furanones and Diaryl-substituted pyrazoles), e.g., COX-2 inhibitors such as valdecoxib, celecoxib, rofecoxib, parecoxib and etoricoxib, the salicylates such as acetylsalicylic acid (aspirin), methyl salicylate and diflunisal, the oxicams such as piroxicam and meloxicam, the sulphonanilides such as nimesulide, the indole acetic acids such as etodolac and other NSAIDs such as mefenamic acid, nabumetone, paracetamol (acetominophen), propacetamol, fenamate, ketamine, dextromethorphan, amantadine, clonidine, dexmedetomidine, gabapentin, magnesium and neostigmine, as well as local anesthetics.

In such embodiments, the opioid analgesic comprises at least one selected from morphine, oxycodone, butorphanol and codeine. In some embodiments, the non-opioid analgesic comprises at least one selected from gabapentin and dextromethorphan.

In some embodiments of the invention, the therapeutic agent of the reservoir is at least one antifungal. In some such embodiments, the antifungal is an azole. Examples of azoles suitable for the invention, without limitation, include amphotericin B, clotrimazole, fluconazole, itraconazole and nystatin.

In some embodiments, the therapeutic agent of the reservoir of the invention is at least one antipruritic (i.e., anti-itch agent). Examples of antihistamines include the first-generation H₁-receptor antagonists including ethylenediamines such as mepyramine (pyrilamine) or antazoline, ethanolamines such as diphenhydramine, carbinoxamine, doxylamine, clemastine, and dimenhydrinate, alkylamines such as pheniramine, chlorphenamine (chlorpheniramine), dexchlorphenamine, brompheniramine, and triprolidine, piperazines such as cyclizine, hydroxyzine, and meclizine, and tricyclics such as promethazine, alimemazine (trimeprazine), cyproheptadine, and azatadine, the second-generation H₁-receptor antagonists including the systemics such as acrivastine, astemizole, cetirizine, loratadine, and mizolastine and topicals such as azelastine, levocabastine and olopatadine, and the third-generation H₁-receptor antagonists including the systemics such as levocetirizine, desloratadine and fexofenadine. In some such embodiments, the anti-itch agent is an antihistamine such as diphenhydramine and its salts (e.g., diphenylhydramine HC1),

In some embodiments, the therapeutic agent of the reservoir of the invention is at least one antibiotic. Examples of antibiotics include, e.g., the aminoglycosides such as amikacin, gentamicin, kanamycin, neomycin, netilmicin, streptomycin and tobramycin, carbacephems such as loracarbef, carbapenems such as ertapenem, imipenem/cilastatin and meropenem, first generation cephalosporins such as cefadroxil, cefazolin and cephalexin, second generation cephalosporins such as cefaclor, cefamandole, cefoxitin, cefprozil and cefuroxime, third generation cephalosporin such as cefixime, cefdinir, cefditoren, cefoperazone, cefotaxime, cefpodoxime, ceftazidime, eftibuten, ceftizoxime and ceftriaxone, fourth generation cephalosporins such as cefepime, glycopeptides such as teicoplanin and vancomycin, macrolides such as azithromycin clarithromycin, dirithromycin, erythromycin, and troleandomycin, monobactam such as aztreonam, penicillins such as amoxicillin, ampicillin, azlocillin, carbenicillin, cloxacillin, dicloxacillin, flucloxacillin, mezlocillin, nafcillin, penicillin, piperacillin and ticarcillin, polypeptides such as bacitracin, colistin and polymyxin b, and quinolones such as ciprofloxacin, enoxacin, gatifloxacin, levofloxacin, lomefloxacin, moxifloxacin, norfloxacin, ofloxacin and trovafloxacin, sulfonamides such as mafenide, prontosil, sulfacetamide, sulfamethizole, sulfanilimide, sulfasalazine, sulfisoxazole, trimethoprim, and trimethoprim-sulfamethoxazole, tetracyclines such as demeclocycline, doxycycline, minocycline, oxytetracycline, and tetracycline, as well as chloramphenicol, clindamycin, ethambutol, fosfomycin, furazolidone, isoniazid, linezolid, metronidazole, nitrofurantoin, pyrazinamide, quinupristin/dalfopristin, rifampin and spectinomycin.

In some embodiments, the therapeutic agent of the reservoir is an antipyretic, e.g., salicylates such as acetylsalicylic acid (aspirin), and paracetamol (acetominophen).

The second layer of the product of the invention is, for example and without limitation, a coating, film, sheet, or pad. The second layer can be of any thickness as the application of the product of the invention dictates. In some embodiments, the second layer of the product is thin, i.e., having little or slight thickness or extent from one surface to its opposite, compared to the overall thickness of the product. In some embodiments, the second layer is a film. In some embodiments, the second layer is a coating. In some embodiments, the second layer is a sheet.

The second layer of the invention is comprised of chitin or a chitin derivative (referred to hereinafter as “chitin”). In some embodiments, the second layer coating, film, sheet, coating and the like is substantially made of chitin. In some embodiments, the entire second layer is substantially contains chitin. In some embodiments, the top face of the second layer contains a chitin. In some embodiments, the second layer is a chitin film, e.g. a laminar film. As used herein, “laminar” means made up of, or arranged in, layers or laminae, which are thin, flat or sheet-like structures. In some embodiments, the second layer is a chitin sheet. In some embodiments, the second layer is a chitin coating. In some embodiments, the second layer is porous, e.g., as a porous film or a porous sheet of chitin.

The chitin second layer of the product of the present invention provides a more efficient form of a dual chitin-therapeutic active drug delivery system than systems that have used mixtures of chitin with an active ingredient substance. For example, the chitin second layer of the present invention, e.g., a chitin film, having a thickness from about 0.1 mm to about 1.0 mm, forms a laminar barrier between the therapeutic active-containing reservoir that can interface directly with the wound the product covers, acting both to separate the therapeutic active from the environment of the product and to provide therapeutic haemostasis to the wound. Release of a therapeutic agent from the reservoir through the chitin second layer to the wound is essentially constant and controlled because of a diffusion gradient formed from different concentrations of therapeutic agent within the reservoir. The chitin film also provides for minimal contact between the chitin and the therapeutic agent, thereby minimizing the potential for diminished activity of the therapeutic agent due to chitin chemical bonding interactions.

A chitin/chitosan film is a more efficient form of chitin/chitosan for drug delivery purposes and is applied differently in the present conception from prior formulae that have used homogenous mixtures of chitin/chitosan with an active ingredient substance. See, for example, U.S. Pat. Nos. 6,897,348 and 6,967,261 which teach a bandage for acute wounds and bums having an antimicrobial agent and a haemostatic agent such as chitosan wherein the antimicrobial agent and the chitosan are chemically linked together. A (0.1-1.0 mm) chitin/chitosan film is designed to form a laminar barrier between an active-substance containing reservoir that can interface directly with the wound, acting both to separate the drug from the environment within the bandage and to provide therapeutic hemostasis to a wound.

Chitin when in physical contact with substances has the potential to impede or inhibit the diffusion of drugs in a mixture composition. The molecular mobility of substances mixed with chitin, in a chitin-substrate medium, depends on possible changes of conformation, steric hindrance and viscosity of the medium. For example, chitin can form a chitosan physical-hydrogel, with different degrees of acetylation, depending on interactions with the hydrophilic/hydrophobic characteristics of drug compounds interacting with the chitin matrix. This allows for the formation of both hydrophilic/hydrophobic interactions and hydrogen bonding. In addition, naturally cationic polysaccharides such as chitin and chitosan have been shown to be effective substances for color removal. This can be attributed to a combination of electrostatic attraction, van der Waals forces, and hydrogen bonding (See e.g., Blackburn, Environ Sci Technol. 2004 Sep. 15; 38(18): 4905-09). Adsorption itself is highly effective and does not involve any additional chemical input or treatment other than the intermixing of chitin and different reactive agents. Such substance-chitin interactions may depend on the charge density of chitosan, modified by the degree of neutralization, the dielectric constant of different solvents related to the composition of the medium, the degree of acetylation, temperature, which can play a role in the interactions responsible for the physical cross-linking, and the molecular mobility of the substance (e.g., therapeutic agent) mixed with chitin (See e.g., Montembault A. et al., Biomaterials, 2005 Mar.; 26(8): 933-43).

Inhibition of a substance's (e.g., a therapeutic agent's) movement to the wound site would be reduced or non-existent with a chitin film (e.g., between about 0.1 mm to about 1 mm thick) since the majority of the substance is not in contact with the chitin, while the interface of the chitin layer (e.g., a film) allows greater diffusion of the substance across e.g., a film barrier, due to the formation of a substance gradient, thereby delivering a greater amount of substance such as therapeutic agent more efficiently than from a chitin-substance mixture.

In some embodiments, the second layer of the product of the invention is laminated, e.g., as a laminated film or laminated sheet. When the second layer is laminated, it has superimposed layers of one or more materials. For example, the second layer can be a laminated film of substantially chitin only, or of chitin and other materials such as film forming polymers, adhesive forming polymers, adhesive releasing polymers, and therapeutic and non-therapeutic agents. A laminated second layer can provide a second layer of increased mechanical strength, durability, and character, and improved quality and character. For example, a laminated second layer can provide for clean, easy and/or effective removal of the product from the wound site. A laminated second layer also can comprise layers of equal dimensions or of unequal dimensions, depending on the composition of each layer and aim of the laminated second layer. For example, a laminated chitin film can involve a substantially chitin layer superimposed by multiples of a second substantially chitin layer that are in alignment across the first chitin layer, whereby the nexus where the multiples of second chitin layers allows for pooling of exudates from the wound.

In some embodiments, the chitin second layer can further comprise an ionic substance such as an ionic form of a therapeutic agent. In some such embodiments, the ionic form of therapeutic agent is of the same therapeutic agent contained in the reservoir of the first internal layer. For example, if the therapeutic agent contained in the reservoir is lidocaine, the chitin second layer can include an ionic form of lidocaine. The ionic substance can be an ionic liquid.

An ionic liquid is a liquid that contains only ions and includes all the molten salts (e.g., sodium chloride) at temperatures higher than 800° C. Ionic liquids also include salts whose melting points are relatively low (below 100° C.) such as salts that melt at room temperature (room-temperature ionic liquids or RTILs), and often those with even lower than room temperature melting points. In ionic liquids, the ions are poorly coordinated, causing its liquid nature, where at least one ion has a delocalized charge and one component is organic, which prevents the formation of a stable crystal lattice.

When a therapeutic agent is an ionic liquid, the agent can be transformed from a solid state (e.g., a powder) to the liquid state. The ionic liquid form of the agent can provide a therapeutic agent of different character than that of its solid state form. For example, an ionic liquid of a therapeutic agent can have enhanced duration regarding efficacy than when not an ionic liquid.

In some embodiments of the present invention, the chitin second layer further comprises an ionic liquid of lidocaine. In some such embodiments, the ionic lidocaine is incorporated in a chitin film. In some such embodiments, the ionic lidocaine has improved anesthetic efficacy than non-ionic lidocaine.

Cross-linking processes known in the art can be used to make chitin into a useable form for the invention, e.g. as a film or porous film form. Suitable polymers forming the second layer film, sheet, coating and the like of the invention are of pharmaceutical grade or “generally recognized as safe” (GRAS) by the U.S. Food and Drug Administration.

Suitable chitins for the second layer of the invention, without limitation, include, chitin, N-acetyl-d-glucosamine, β-1,4-poly-d-glucosamine chitosan, β-1,4-poly-d-glucosamine/poliglusam chitosan, deacetylchitin/poliglusam, chitosan malate, chitosan oligosaccharide, chitosan niacinamide ascorbate salt, D-glucosamine hydrochloride, D-glucosamine sulfate 2KCl, D-glucosamine sulfate 2NaCl, and deacetylchitin; and embraces naturally and synthetic chitin and its derivatives, including poly(N-acetylgluosamine) and its epimer, poly (N-acetylgalactosamine). In some embodiments, the chitin of the second layer is at least one of chitin, N-acetyl-d-glucosamine, β-1,4-poly-d-glucosamine chitosan, β-1,4-poly-d-glucosamine/poliglusam chitosan, deacetylchitin/poliglusam, chitosan malate, chitosan oligosaccharide, D-glucosamine hydrochloride, D-glucosamine sulfate 2KCl, D-glucosamine sulfate 2NaC or deacetylchitin.In some embodiments, the chitin is chitin. In some embodiments, the chitin is at least one β-1,4-poly-d-glucosamine chitosan, β-1,4-poly-d-glucosamine/poliglusam chitosan, deacetylchitin/poliglusam, chitosan malate, or chitosan oligosaccharide. In some embodiments, the chitin is deacetylchitin.

Chitin is a white to off-white, insoluble, linear homopolymer composed of N-acetylglucosamine residues in β-(1,4) linkage that it is widely distributed, forming the principal constituent of arthropod exoskeletons, and found in some plants, particularly fungi. Suitable sources of chitin are from lobsters, shrimp, other crustacea and fungi. Its derivative chitosan is a non toxic, biodegradable polymer of high molecular weight, similar in molecular structure to cellulose. After cellulose, it is the most common polysaccharide found in nature. Like cellulose, chitin is a fiber. Chitosan is obtained by removing enough acetyl groups from the chitin molecule to be soluble in most diluted acids. This process, called deacetylation, releases amine groups and gives the chitosan a cationic characteristic. This is especially interesting in an acid environment where the majority of polysaccharides are usually neutral or negatively charged.

The chitin second layer of the invention may vary in molecular weight or degree of deacetylation.

Chitin and chitosan derivatives are used as excipients and drug carriers in the pharmaceutical field. Chitosan is used as an excipient in oral dosage forms. Films prepared using chitin or chitosan have been developed as wound dressings, oral mucoadhesives and water-resisting adhesives by virtue of their release characteristics and adhesion.

In some embodiments, the product of the invention includes a second layer that is a chitin film that contacts a wound as a protective, haemostatic film, which allows diffusion of a therapeutic agent (e.g., lidocaine) through the film and into the wound. The chitin second layer inhibits bacteria, has good histocompatibility and excellent ability for passing through mist, transferring exudates and absorbing exudates.

In some embodiments , the product of the invention is a bandage that contains an analgesic such as lidocaine in a first internal layer and has as a second internal layer a chitin film thereby providing a complete bandage for treating wounds.

Another aspect of the present invention relates to a method of treating a wound using the product of the invention. In some embodiments, the method involves affixing to an area surrounding the wound the product of the invention thereby, covering the wound. In some such embodiments, the second layer of the product is in close proximity to the wound. In some such embodiments, the top face of the second layer is in close proximity to the wound.

In some embodiments of the method of treating a wound, the product of the invention has an analgesic such as lidocaine in the reservoir and a porous chitosan film where the chitosan film is placed in contact with a integumental wound thereby forming a coagulum to prevent bleeding while allowing the therapeutic agent to pass freely from the reservoir through the chitosan film to the wound.

As used herein, “wound,” refers to any abrasion, burn, cut, incision, irritation, laceration, puncture, scrape, scratch, or the like associated with an integument (i.e., an outer protective covering such as the skin of an animal), where the integument is not in its substantially normal state of being. The wound can be of any degree of severity, size, scope or the like, or of any length in existence. The product of the invention, thereof, has both human and veterinarian utility, and can be administered to animals of the aves, reptilia, or mammalia classes. In some embodiments of the method of the present invention, the product is affixed to animals selected from birds, reptiles or mammals. In some embodiments of the methods of the present invention, the animal is a mammal. In some such embodiments, the mammal is human. In some such embodiments, the mammal is nonhuman. Non-human mammals for which the product of the invention is suitable include, for example, dogs, cats, other domesticated mammals, (commonly referred to as “pets”), cows, cattle, pigs, horses, sheep, goats and other farm, show and commercially- or economically-valuable animals.

Those skilled in the art will appreciate that numerous changes and modifications may be made to the embodiments of the invention and that such changes and modifications may be made without departing from the spirit of the invention. It is therefore intended that the appended claims cover all such equivalent variations as falling within the true spirit and scope of the invention.

It is further intended that each of the patents, applications, printed publications, and other published documents, including books, mentioned or referred to in this specification be herein incorporated by reference in their entirety.

What is claimed is:

1. A product comprising:
a. a base layer;
b. a first internal layer; and
c. a second internal layer,
wherein:
the base layer has an exterior surface and an interior surface;
the first internal layer comprises
i. a top face;
ii. a bottom face;
iii. an adhesive; and
iv. a reservoir;
wherein:
the bottom face of the first internal layer adheres to the interior surface of the base layer by virtue of the adhesive;
the reservoir is housed between the top and bottom faces of the first internal layer and comprises at least one therapeutic agent; and
the second internal layer comprises:
i. a chitin or a chitin derivative;
ii. a top face; and
iii. a bottom face,
wherein the bottom face of the second internal layer adheres to the top face of the first internal layer.

2. The product of claim 1, wherein the reservoir comprises at least one therapeutic agent selected from the group consisting of anesthetics, analgesics, antifungals, antibiotics, antipruritics, and antipyretics.

3. The product of claim 2, wherein the reservoir further comprises a support matrix that contains the therapeutic agent.

4. The product of claim 3, wherein the support matrix comprises at least one gel, emulsion, collodion, or fibrous substance.

5. The product of claim 4, wherein the support matrix is a gel.

6. The product of claim 5, wherein the gel comprises at least one silicone polymer, gelatin, pectin, collagen, polyacrylamide, ethylene dimethacrylate polyethylene glycol dimethacrylate, glycol dimethacrylate, or acemannan.

7. The product of claim 4, wherein the fibrous substance comprises at least one cellulose, cotton, modal, nylon, rayon, polyester fiber, acrylic fiber, aramide fiber, elastane or wool.

8. The product of claim 4, wherein the reservoir further comprises at least one stabilizer, emulsifier, thickening agent, antioxidant, or control release agent.

9. The product of claim 2, wherein the reservoir comprises at least one anesthetic.

10. The product of claim 9, wherein the anesthetic comprises at least one local anesthetic or a pharmaceutical salt thereof.

11. The product of claim 10, wherein the local anesthetic comprises at least one selected from lidocaine, benzocaine, procaine, cocaine, eucaine, prilocaine, tetracaine, oxybupricaine or phenacaine.

12. The product of claim 11, wherein the anesthetic is lidocaine.

13. The product of claim 12, wherein the anesthetic comprises about 1% to about 10% of the total therapeutic agent.

14. The product of claim 2, wherein the reservoir comprises at least one opioid or non-opioid analgesic.

15. The product of claim 14, wherein the opioid analgesic comprises at least one compound selected from the group consisting of morphine, codeine, hydrocodone, hydromorphone, oxycodone, oxymorphone, nalbuphine, naloxone, naltrexone, buprenorphine, butorphanol, etorphine, methadone, levo-alphacetylmethadol (LAAM), pethidine (meperidine), fentanyl, alfentanil, sufentanil, remifentanil, ketobemidone, carfentanyl, propoxyphene, dextropropoxyphene, dextromoramide, bezitramide, piritramide, pentazocine, phenazocine, buprenorphine, butorphanol, nalbufine, dezocine, etorphine, tilidine, tramadol, loperamide and diphenoxylate.

16. The product of claim 14, wherein the non-opioid analgesic comprises at least one compound selected from the group consisting of a nonsteroidal anti-inflammatory, a salicylate, an oxicam, a sulphonanilide, an indole acetic acid, mefenamic acid, nabumetone, paracetamol, propacetamol, fenamate, ketamine, dextromethorphan, amantadine, clonidine, dexmedetomidine, gabapentin, magnesium and neostigmine.

17. A method of treating a wound comprising affixing to an area surrounding the wound the product of claim 1 thereby, covering the wound.

18. The method of claim 20, wherein the wound to an integument comprises a abrasion, burn, cut, incision, irritation, laceration, puncture, scrape or scratch.
